(19) United States
(12) Patent Application Publication
(10) Pub. No.: US 2012/0245528 A1
(43) Pub. Date: Sep. 27, 2012

(54) CATHETER WITH POROUS CUFF

(75) Inventors: Raymond Lareau, Westford, MA (US); Benjamin Bell, Shrewsbury, MA (US); Mark Girard, Medway, MA (US)

(73) Assignee: Navilyst Medical, Inc., Marlboro, MA (US)

(21) Appl. No.: 13/053,386

(22) Filed: Mar. 22, 2011

(51) Int. Cl. A61M 25/04

(52) U.S. Cl. 604/175

(57) ABSTRACT

Disclosed is a catheter that includes a porous element. In one embodiment, the porous element comprises the same polymeric material as the catheter shaft. In another embodiment, the porous element comprises a porous polyurethane material.

### FIELD OF THE INVENTION

The present invention relates to tunneled catheters that make use of one or more porous cuffs to anchor the catheter under a patient's skin, and/or to minimize the risk of infection.

### BACKGROUND

There are a number of implantable medical devices, such as indwelling catheters, that are used for the repeated and prolonged access to a patient's vascular system or other bodily conduits. Such devices include peripherally-inserted central catheters (“PICC's”), central venous catheters (“CVC's”), dialysis catheters, implantable ports, and midline infusion catheters. These devices are typically implanted into a patient for an extended period of time to allow for multiple treatments, such as the delivery of therapeutic agents or dialysis treatments. Use of such devices eliminates the need for multiple placements of single-use devices, thus reducing the risk of infection and placement complications, and reducing the overall cost of patient care. Examples of such implantable medical devices include Vaxcel® PICC's and ports, Xcela® PICC's and ports, and Vaxcel® Plus Chronic Dialysis catheters (all from Navilyst Medical, Inc., Marlborough, Mass.).

In many cases, indwelling catheters are used as “tunneled” catheters that are placed into a percutaneous incision, for example in the chest, and then threaded through a tunnel of tissue before entering a blood vessel. The tissue “tunnel” helps to anchor the catheter and to prevent infection from spreading into the blood vessel. To further anchor such catheters and to prevent infections, a cuff may be placed around the catheter shaft at or distal to the site of catheter entry. Such cuffs are typically made from polyester, such as in the form of DACRON® (Invista North America S.A.R.L. Corporation, Wilmington, Del.) fibers, and their porous and/or fibrous structure promotes the ingrowth of surrounding tissue and the consequent anchoring of the catheter within the tissue.

One potential limitation of currently available cuffed catheters is that the catheter shaft material is typically made from polyurethane or some other polymeric material that is different from the cuff material. Cuffs are therefore typically applied to catheter shafts by adhesive materials. As a result, the strength of the bond between the cuff and the catheter shaft is limited by the strength of the adhesive material used to join them. In addition, the materials and structures of current cuff materials are such that their internal pores are flexible and collapsible, and are therefore generally unable to hold their shape in a way that allows for the containment of therapeutic agents. One additional limitation of current cuff structures is that it is not possible to tailor the porosity in the cuff to allow for specific applications; for example, a high degree of porosity and/or larger pores for enhanced tissue ingrowth for longer-term applications, versus a lower degree of porosity and/or smaller pores for limited tissue ingrowth for shorter-term applications.

### SUMMARY OF THE INVENTION

In one aspect, the present invention includes a catheter that comprises a shaft having proximal and distal ends, wherein the shaft comprises a first polymeric material. The catheter includes a first opening at the proximal end of the shaft and a second opening at the distal end, and a lumen extending between the first and second openings. The catheter further includes a porous element disposed about a portion of the catheter shaft, wherein the porous element comprises the first polymeric material and is preferably positioned closer to the proximal end of the shaft than the distal end of the shaft.

In another aspect, the present invention includes a catheter that comprises a shaft having proximal and distal ends, wherein the shaft comprises a first polymeric material. The catheter includes a first opening at the proximal end of the shaft and a second opening at the distal end, and a lumen extending between the first and second openings. The catheter further includes a porous element disposed about a portion of the catheter shaft, wherein the porous element comprises a porous polyurethane material.

In another aspect, the present invention comprises a method of treating a patient using the catheters of the present invention.

In yet another aspect, the present invention comprises a kit that includes one or more catheters of the present invention.

### DESCRIPTION OF THE DRAWINGS

FIG. 1 is a top view of a catheter, according to an embodiment of the present invention.

FIG. 2 is a perspective view of a tubular porous material used to provide a catheter cuff, in accordance with an embodiment of the present invention.

FIG. 3 is a perspective view of a porous sheet material used to provide a catheter cuff, in accordance with an embodiment of the present invention.

FIG. 4 is a perspective view of a tubular porous material used to provide a catheter cuff, in accordance with an embodiment of the present invention.

### DESCRIPTION OF THE PREFERRED EMBODIMENTS

The present invention provides catheters, and more particularly indwelling tunneled catheters, that may reside in the vasculature or other bodily lumens of patients for prolonged periods of time. As used herein, the term “indwelling catheter” is intended to include any flexible tube that is placed and left in the body over an extended time period. The catheters of the present invention include cuffs characterized by materials and structures that yield enhanced bonding between cuff and catheter shaft, may be customized for predetermined levels of tissue ingrowth, and allow for the delivery of therapeutic agents.

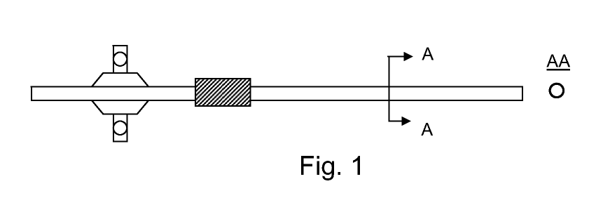

A top view of a catheter according to an embodiment of the present invention is shown in FIG. 1. Although an exemplary indwelling catheter is shown in the figures included herein, it should be appreciated that the present invention is applicable to any catheter device that resides within a patient for an extended period of time. Such catheters include PICC's, CVC's, dialysis catheters, infusion catheters, drainage catheters, and any other tubular devices that are inserted into bodily lumens or organs for the delivery and/or withdrawal of fluids.

The catheter 100 illustrated in FIG. 1 includes a shaft 101 that includes a proximal end 110, a distal end 120, and a tubular sidewall 130 extending between the proximal end 110 and distal end 120. A portion of the catheter 100 distal from the proximal end 110 is configured to remain outside of a patient's body when the catheter 100 is in use. For example, the proximal end 110 optionally includes suture wings 111 that are attachable to the skin of a patient via sutures or the like. The proximal end 110 also optionally includes a luer fitting (not shown) for connection to a fluid source, such as medications, saline, nutrients, and blood. The distal end 120 is configured to be inserted into a patient to reside within any suitable bodily structure, such as a bodily lumen (e.g., a blood vessel, the urethra, the ureter, the esophagus, or the colon) or an organ (e.g., the kidney, the heart, or the stomach). The tubular sidewall 130 extending between the proximal end 110 and distal end 120 defines a lumen 140 for the passage of such fluids to or from the patient.

The catheter 100 includes a porous element or cuff 150 that extends at least partially around, and preferably completely around, the tubular sidewall 130. When the catheter 100 is placed within a patient, the cuff should be at or near the location where the catheter 100 extends through the patient's skin such that at least a portion of the cuff extends into the patient's tissue. The use of cuffs as part of indwelling catheters is known to help secure the catheter in place and to help form an infection barrier.

The materials used to fabricate the catheters of the present invention are any suitable polymeric materials as are known in the art, such as thermoplastic polyurethanes, nylons, polyether block amides, ethylene vinyl acetate, silicones, polyolefin elastomers, styrenic elastomers, and polyester elastomers. The catheters are preferably manufactured by extrusion fabrication techniques, as are known in the art.

In contrast to known woven catheter cuffs that are made from materials that are dissimilar to the catheter wall material, the porous cuffs of the present invention are made from materials that are the same as, or substantially similar to, the catheter wall material. For example, in an embodiment of the present invention, the tubular wall 130 and the porous cuff 150 are made from polyurethane, and more particularly, polyether or polycarbonate polyurethanes, such as Carbothane® or Tecoflex® (The Lubrizol Corporation, Wickliffe, Ohio). In other embodiments, the tubular wall 130 and the porous cuff 150 are made from a silicone material. The similar material used for both the catheter wall and cuff materials are believed by the inventor to result in improved bonding between these two components, and also allows for a porous cuff structure that facilitates the incorporation of therapeutic agents for delivery to surrounding body tissue.

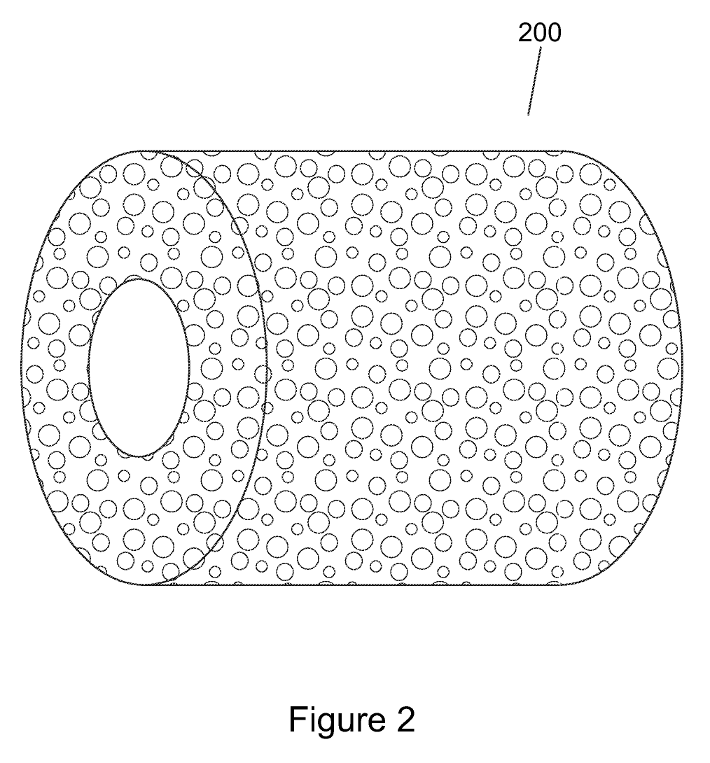

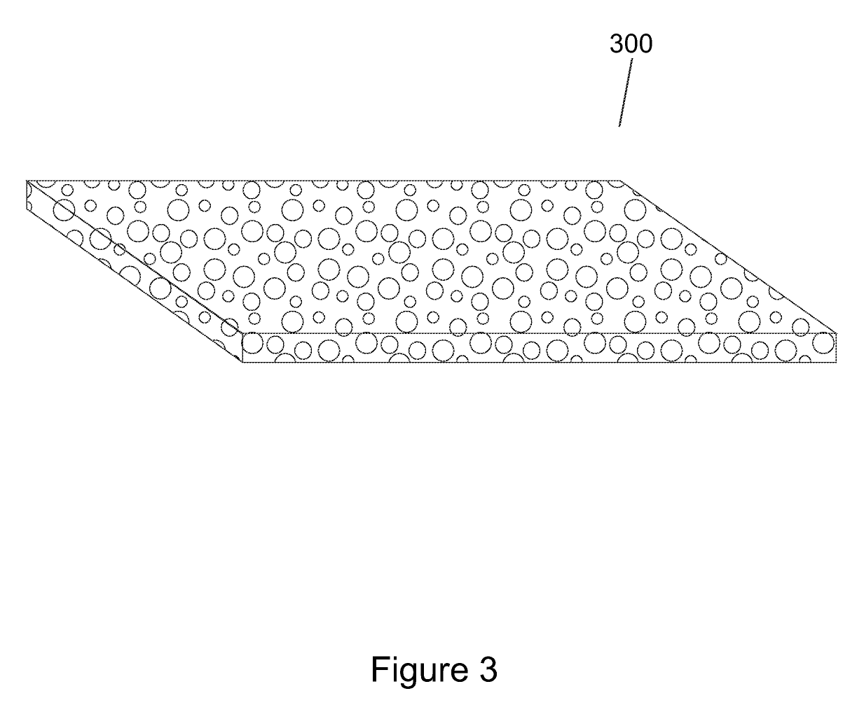

The porous nature of the cuffs used the present invention is formed by any suitable technique, such as by rapid solvent evaporation, free-drying, or the introduction of wax or water-soluble materials in the polymer mixture followed by applicable post-processing techniques. The cuff material is fabricated in any suitable configuration, such as a tube 200 as shown in FIG. 2 or a sheet 300 as shown in FIG. 3, which are subsequently cut to desired dimensions for placement around the tubular wall 130 to form porous element or cuff 150. As shown in FIGS. 2 and 3, the material used to form the cuff 150 is preferably porous, with an open-pore construction to provide the mechanical properties and optional drug release characteristics described herein.

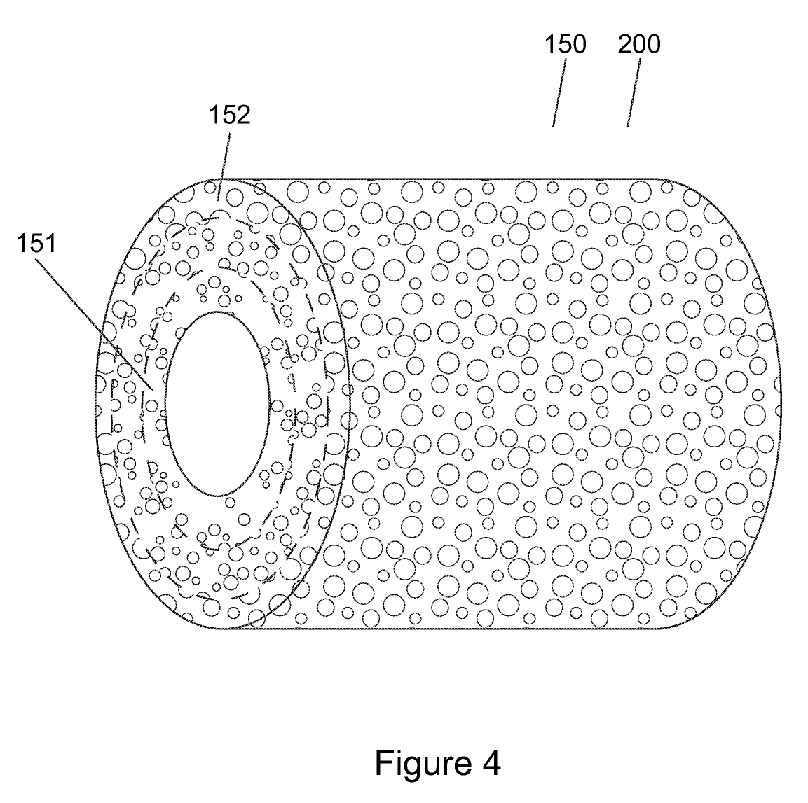

In a preferred embodiment, polyurethane containing a low concentration of solvent is used to coat a mandrel, and the solvent is rapidly removed from the urethane solution by flashing or evaporation to yield a porous sleeve structure, as shown removed from the mandrel in FIG. 2. The thickness of the sleeve can be increased by performing multiple coating and solvent removal cycles. As is known in the art, the pore structure can be altered or tailored by controlling the solvent concentration, solvent type, solution viscosity, and the means by which solvent is removed. For example, in one preferred embodiment illustrated in FIG. 4, the pores within the cuff 150 are smaller and/or are found at lower volume percentages at or near the “inner” surface 151 that joins with the tubular wall 130 when compared with the “outer” surface 152 that is exposed to surrounding tissue, to thereby provide a structure that enables a strong bond with the tubular wall 130 yet provides a highly porous structure for tissue ingrowth and/or drug delivery.

In alternate embodiments, sheets of cuff material are prepared using a flat plate. The plate can be dipped into a solution of cuff material, or the solution can be poured onto the plate, followed by removal of solvent from the solution by flashing or evaporation. The pore structure within sheets can be altered or tailored by controlling solvent concentration, solvent type, solution viscosity, and the means by which solvent is removed, as described above.

The cuff material, either in sheet or tubular form, is joined to the surface of the tubular wall 130 of the shaft 101 by any suitable technique to form the cuff 150. In a preferred embodiment, the cuff material is joined to the tubular wall 130 in the absence of any adhesive or other joining material. Examples of preferred joining techniques are thermal and solvent bonding. The choice of solvent will vary depending on the choice of polymer used to form the cuff. As non-limiting examples, tetrahydrofuran, isopropanol, and N-methylpyrrolidone can be used as solvents for bonding. In alternate embodiments, the cuff may be provided during the manufacture of the tubular wall 130 in any suitable technique, such as, for example, direct molding. In certain embodiments, the cuff is formed directly onto the catheter wall by masking off the surface of the catheter, and applying a polymer/solvent solution to the exposed portion of the catheter wall.

In one embodiment, the cuff material is fabricated into a sheet 300 as shown in FIG. 3, which is provided in a kit together with the catheter 100 (without a cuff 150) to the end user, such as the health care provider responsible for implanting the catheter 100 into a patient. The end user will be able to cut or otherwise tailor the size and/or configuration of the sheet 300 to address and meet specific patient needs. The kit preferably includes means to attach the sheet 300 to the tubular wall 130 of the shaft 101, such as an adhesive backing, adhesive material that is heat, light, or chemically activated, or a solvent useful for solvent bonding the sheet 300 to the tubular wall 130.

One or more therapeutic agents are optionally added to the porous cuff 150 prior to insertion into the patient. Examples of such therapeutic agents include those that help to prevent infection and to promote tissue ingrowth; such as antibiotics, antimicrobals, and antiviral agents, including minocycline, rifampin, chlorohexadine, sulfadiazine, penicillin, tetracycline, oxytetracycline, metacycline, doxycycline, minocycline, fradiomycin sulfate, erythromycin, chloramphenicol, methyl hydroxybenzoate, propyl hydroxybenzoate, chlorocresol, benzalkonium chlorides, nitrofurazone, nystatin, sulfacetamide, clotriamazole, or the like, or a combination thereof; and/or cell growth promoters such as growth factors, transcriptional activators, and translational promoters.

It will be apparent to those skilled in the art that various modifications and variations can be made in the structure and methodology of the present invention. Thus, it is intended that the present invention cover such modifications and variations provided that they come within the scope of the appended claims and their equivalents.

We claim:

1. A catheter, comprising:
a shaft comprising a proximal end and a distal end, wherein said shaft comprises a first polymeric material;
a first opening at said proximal end and a second opening at said distal end;
a lumen extending between said first and second opening;
a porous element disposed about a portion of said shaft, wherein said porous element comprises the first polymeric material and is positioned closer to the proximal end of the shaft than the distal end of the shaft.

2. The catheter of claim 1, wherein said catheter does not include adhesive between said shaft and said porous element.

3. The catheter of claim 1, wherein said porous element is thermally bonded to said shaft.

4. The catheter of claim 1, wherein said porous element is solvent bonded to said shaft.

5. The catheter of claim 1, wherein said porous element is molded onto said shaft.

6. The catheter of claim 1, wherein said first polymeric material is polyurethane.

7. The catheter of claim 1, wherein said porous element comprises a therapeutic agent.

8. The catheter of claim 7, wherein said therapeutic agent comprises an antimicrobial agent.

9. The catheter of claim 7, wherein said therapeutic agent comprises a growth factor.

10. The catheter of claim 1, wherein said porous element comprises an outer surface adapted to contact tissue.

11. The catheter of claim 10, wherein said porous element comprises a first level of porosity at an interface between said shaft and said porous element, and a second level of porosity at said outer surface, said first level of porosity being less than said second level of porosity.

12. A catheter, comprising:
a shaft comprising a proximal end and a distal end, wherein said shaft comprises a first polymeric material;
a first opening at said proximal end and a second opening at said distal end;
a lumen extending between said first and second opening;
a porous element disposed about a portion of said shaft, wherein said porous element comprises a porous polyurethane material.

13. The catheter of claim 12, wherein said catheter does not include adhesive between said shaft and said porous element.

14. The catheter of claim 12, wherein said porous element is thermally bonded to said shaft.

15. The catheter of claim 12, wherein said porous element is solvent bonded to said shaft.

16. The catheter of claim 12, wherein said porous element is molded onto said shaft.

17. The catheter of claim 12, wherein said first polymeric material is polyurethane.

18. The catheter of claim 12, wherein said porous element comprises a therapeutic agent.

19. The catheter of claim 18, wherein said therapeutic agent comprises an antimicrobial agent.

20. The catheter of claim 18, wherein said therapeutic agent comprises a growth factor.

21. The catheter of claim 12, wherein said porous element comprises an outer surface adapted to contact tissue.

22. The catheter of claim 21, wherein said porous element comprises a first level of porosity at an interface between said shaft and said porous element, and a second level of porosity at said outer surface, said first level of porosity being less than said second level of porosity.
